(19) United States
(12) Patent Application Publication
(10) Pub. No.: US 2023/0399102 A1
(43) Pub. Date: Dec. 14, 2023

(54) SYSTEM AND METHOD FOR PASSENGER COMMUNICATION IN A VEHICLE

(71) Applicant: APIOS LIMITED, Chichester, West Sussex (GB)

(72) Inventors: David Robert Thomas, Chichester (GB); James Henry Daniel Woodhead, Leeds (GB); Phil Hall, Wooten Wawen (GB); Tom Michael Walker, Chester (GB); John Michael Wright, Isle of Wright (GB)

(21) Appl. No.: 18/034,745

(22) PCT Filed: Oct. 28, 2021
(86) PCT No.: PCT/GB2021/052797

§ 371 Date: May 1, 2023

(30) Foreign Application Priority Data
Oct. 29, 2020 (GB) 2017144.3

(51) Int. Cl. B64D 11/00; G06F 40/58

(52) U.S. Cl. CPC B64D 11/0015; G06F 40/58

(57) ABSTRACT

A method for communicating with a passenger making a journey in a vehicle is provided, the vehicle having a broadcast system for transmitting information to one or more passengers, the passenger having and operating a Portable Electronic Device (PED), the PED operable to receive information from the broadcast system of the vehicle and to display information content to the passenger; the method comprising transmitting information to the PED of one or more passengers within the vehicle; wherein the PED displays information content derived from the information transmitted to the PED. A communication system for performing the method and a vehicle comprising the communication system are also provided.

The present invention relates to systems for communication with passengers in a vehicle and to vehicles comprising the same. The present invention also relates to methods for communicating with passengers in a vehicle. In one aspect, the present invention relates to public transport passenger entertainment.

It is common for passenger carrying vehicles, such as aircraft, ferries, coaches, trains and buses, to be provided with seat-based entertainment systems. Generally, there is a need for the operators of passenger carrying vehicles to communicate with passengers. For example, regulations for passenger carrying vehicles require passengers to be informed of any safety or operational information before and during their transit. As will be readily apparent, there is also a need to communicate with passengers in the case of an emergency involving part or all of the vehicle. It is common practice to equip passenger carrying vehicles with systems allowing announcements to be made to the general passenger carrying area over a public announcement system. However, it is common practice to also transmit information, such as announcements and the like, to passengers by means of the seat-based entertainment systems. This is particularly important when users of the seat-based entertainment systems would be unable to hear announcements being made on the general public announcement system.

Further, the majority of passengers travelling in vehicles possess a Portable Electronic Device (PED) and frequently use their PED during transit. A passenger consuming content on their PED may be unable to hear or see announcements being made either to the general passenger carrying area or to a seat-based entertainment system.

There is a need for a method to enable passengers consuming content on their PED to be notified of safety and operational information. There is also a need to provide passenger carrying vehicles, such as those used for public transport, with an economic installation of seat back and overhead video systems by reducing installation costs associated with installation, maintenance and amortisation, preferably enabling the passenger to consume media more conveniently, for example on a larger screen at a more comfortable viewing angle.

Portable Electronic Devices (PEDs), such as mobile telephones, tablet computers and laptop computers, store and access content for the user to consume. To allow the user to view and interact with the content, the PED typically provides a display, communications, interfacing and computing resources. Generally, therefore, the systems and operation of the PED replicate those within seat-based entertainment systems of passenger carrying vehicles, such as aircraft, ferries, coaches, trains and buses.

The increased reliance on connectivity, be it social or work related, while travelling has resulted in increased use of PEDs within the public transport system; a trend that will increase.

However, this increased use of PEDs has a potential negative impact on operational safety as passengers engaged with their PEDs are highly likely to be ignorant of any safety or operational announcements being made by the crew. Frequent travelers, particularly those using air travel, will no doubt be aware of fellow travelers ignoring the safety broadcasts instead while their own personal devices to read emails, to listen to music, to watch video based media.

There has now been found a way to use a PED in a controlled way within a vehicle which enables the owner of the PED to consume media in a more convenient manner, for example on a larger screen at a more comfortable viewing angle, while ensuring the safety protocols and procedures of the vehicle, discussed hereinbefore, to be followed.

There is now provided a method for enabling a passenger in a vehicle, for example a commercial transport vehicle, to consume content provided by the carry-on Portable Electronic Device (PED).

The present invention provides the use of passenger carry on devices (Portable Electronic Devices or PEDs), such as Smartphones, tablets and other portable computers, to be used as an Information Host in a passenger vehicle, such as a public transport vehicle, by enabling the conveyance of crew based service announcements to the PED ensuring a passenger is fully aware of safety and operational announcements as required by regulatory authorities.

In a first aspect, the present invention provides a method for communicating with a passenger making a journey in a vehicle, the vehicle having communication system comprising a broadcast system for transmitting information to one or more passengers, the passenger having and operating a Portable Electronic Device (PED), the PED operable to receive information from the broadcast system of the vehicle and to display information content to the passenger;
the method comprising broadcasting information to the PED of one or more passengers within the vehicle;
wherein the PED displays information content derived from the information transmitted to the PED.

In a further aspect, the present invention provides a Portable Electronic Device (PED) operable to receive transmitted information from a broadcast system of a vehicle in which the user of the PED is travelling and to display information content to the user.

In a further aspect, the present invention provides a communication system for a passenger vehicle, the communication system comprising a broadcast system for transmitting information to one or more passengers, the one or more passengers having and operating a Portable Electronic Device (PED), the PED operable to receive transmitted information from the broadcast system of the vehicle and to display information content to the one or more passengers.

In a still further aspect, the present invention provides a passenger vehicle for carrying one or more passengers, the vehicle comprising a communication system comprising a broadcast system for transmitting information to one or more passengers, the one or more passengers having and operating a Portable Electronic Device (PED), the PED operable to receive transmitted information from the broadcast system of the vehicle and to display information content to the one or more passengers.

Travel is an everyday part of life; be it on a train, bus, ferry or aircraft. Each sector is supported by multiple carriers all competing and vying for the same business. Longer journey durations result in increased demands by the passengers to remain connected to the internet. These demands may include business services, such as email, web browsing, instant messaging and tele-conferences. In other areas, passengers may want to relax and be entertained either by media present on their own PED or by streaming new content.

In the present specification, references to a ‘Portable Electronic Device’ or ‘PED’ are to any piece of lightweight, electrically-powered equipment, especially consumer electronic devices, capable of communications, data processing and/or utility, in particular electronic devices having the capability to store, record, and/or transmit text, images/video, or audio data. Examples of PEDs include mobile telephones, especially so-called smart telephones, tablets, portable computers, for example laptops and notebook computers, and the like.

In the present specification, references to a ‘passenger vehicle’ are to any vehicle constructed or adapted for the transport of people. The vehicle may be a land-based vehicle, for example operating on roads, such as cars, coaches and buses, or rails, such as trains and trams; a water-borne craft, such as a ship, boat, ferry or a hovercraft; an air-borne craft, such as an aeroplane or helicopter; or a vehicle operating in the upper atmosphere and/or in space outside the atmosphere, for example passenger space craft. A preferred passenger vehicle is an air-borne craft, in particular an aeroplane.

The present invention is of particular advantage in passenger vehicles where space within the passenger portion of the vehicle is limited or restricted and/or the weight of the vehicle is a key factor. Examples include air-borne craft, such as passenger aeroplanes, where space and weight are both significant factors, and craft for use in the upper atmosphere or in space, again where both space and weight are severely constrained.

The present invention is particularly advantageous when used in passenger vehicles in which information is frequently transmitted to passengers and/or it is required to provide passengers with information from time to time, for example for safety reasons. Examples include trains, buses and coaches, and flying craft, in particular aeroplanes.

The present invention operates to allow information to be communicated from the operator of the passenger vehicle to one or more passengers travelling in the vehicle. In this respect, references to a passenger travelling in or within a vehicle include passengers about to board the vehicle and passengers about to disembark from the vehicle.

The vehicle comprises a communication system. The communication system comprises a broadcast system for transmitting information to one or more passengers. Generally, it is well known for vehicles to comprise systems allowing information to be provided to passengers, for example from operators or crew members of the vehicle. For example, it is known to provide information to passengers aurally using a public announcement (PA) system having speakers located within the passenger area of the vehicle and/or to individual seat installations. It is also known to provide information by audio and audio/visual displays on the entertainment system of a seat installation.

In the present invention, the broadcast system is configured to transmit information to the PED of one or more passengers. The broadcast system may transmit information to the PEDs of passengers in any suitable manner or combination of manners.

In one embodiment, the broadcast system transmits information to the PEDs by a hard wired connection or network within the vehicle. In this case, the passenger is required to connect their PED to the hard wire network of the vehicle, for example by a suitable cable. The broadcast display may be triggered by signals carried over any hard wired connection. The hard wired connection to the PED may be used to transmit both information and electrical power to the PED.

Alternatively, or in addition, the broadcast system transmits information to the PEDs by a wireless transmission. Preferably, the broadcast system operates to establish a wireless network within and around the vehicle, to which the PEDs of passengers are connected. Suitable wireless formats are known in the art and include wireless networks complying with the Institute of Electrical and Electronics Engineers (IEEE) standards, for example the wireless internet (WiFi) family of wireless network protocols, based on the IEEE 802.11 standard. Other wireless formats include short range wireless standards, such as Bluetooth, and near field communications (NFC).

The present invention employs the PEDs of passengers. The PED communicates with the broadcast system of the communication system, including receiving information from the broadcast system regarding information to be displayed to the passenger owning the PED. The PED is programmed with software enabling signals received from the broadcast system of the vehicle to be processed and actioned by the PED. In one preferred embodiment, the PED is provided with mobile application software, herein referred to as an ‘App’. The software may be provided to the PED in any suitable manner, for example by way of a download from the internet, as is well known in the art. In one embodiment, the software provided to the PED is dedicated to the operator of the passenger vehicle and operates only to communicate with the broadcast systems of a single vehicle operator, for example the vehicles of a single airline company.

In operation of the present invention, information is transmitted by the communication system to the PED of one or more passengers and information content is displayed to each passenger by the PED. The information content displayed to the passenger by the PED is derived from the information transmitted by the communication system. For example, the information content displayed may be contained in the transmitted information and/or the information content displayed may be produced locally by the PED, for example by retrieval from the memory of the PED.

The information may be communicated to the one or more passengers for content to be displayed by the PED of each passenger in any suitable format, for example as an audio message, a visual message or a combination of formats.

The information content to be communicated and displayed to the one or more passengers may be in a pre-recorded form stored on a data storage device in the vehicle. Suitable data storage devices are known in the art and include any medium that allows data to be stored and retrieved when required to be communicated to passengers. In this embodiment, when information content is to be communicated and displayed to one or more passengers, the information is retrieved from the data storage device and transmitted to the PED of the one or more passengers. Retrieval and transmission of the information may be triggered manually by a member of the crew of the vehicle or may be triggered automatically, for example by one or more systems of the vehicle and/or an event involving the vehicle or a part thereof.

Alternatively, the information content to be displayed may be provided by an operator or a member of the crew of the vehicle, for example orally by being spoken and/or by means of signs. The information generated in this way is transmitted contemporaneously to the PEDs of the one or more passengers. The broadcast may be live and delivered directly by any member of the passenger vehicle crew.

In one embodiment, live announcements may be processed, using speech-to-text software generally known in the art, to convert a voice-based announcement in realtime to be displayed in a text format on the display of the PED, the seat back display and/or any other display within the confines of the passenger carrying vehicle. The speech-to-text software may be stored and operated by the communication system of the vehicle, in which case the information transmitted to the PED of the passenger contains the message to be displayed in a text format. Alternatively, the speech-to-text software may be stored and operated by the PED. In this case, the voice-based information is transmitted to the PED, which then converts the information into a text message for display.

In a further alternative, the information content to be displayed to the one or more passengers may be stored within the PED of each passenger. Alternatively, the information to be displayed may be stored in a data memory remote from the PED and associated with a display device, such as a monitor, which is employed by the PED to display information to the passenger. For example, the information may be stored in the form of a pre-determined message to be displayed to the passenger when required, such as for safety reasons. When it is required to display information content to the passenger, a signal may be transmitted by the operator or a member of the crew of the vehicle to the PED of the passenger instructing the PED to display the appropriate information content to the passenger.

The pre-determined messages stored in the PED or the remote storage device may be in a variety of formats. For example, the pre-determined messages may be stored in a plurality of different languages. The pre-determined messages may be arranged to be communicated in the natural language of the user/owner of the PED.

The pre-determined messages displayed to the passenger by means of the PED may be text based, voice based or moving image (video or animation) based.

In one embodiment, the broadcast display may be triggered by an ‘over the air’ signal received by the PED causing one or more pre-determined messages to be displayed on the PED.

The PED may be programmed to translate messages or announcements made by the operators or crew member of the vehicle and transmitted to the PED of the passenger. For example, the passenger may select their native or preferred language to receive displayed information. The PED may translate an incoming message in one language into the language selected by the passenger.

Alternatively or in addition, the PED may convert the format of incoming information received from the broadcast system of the vehicle before displaying information content to the passenger. For example, the PED may receive information relating to an audio message and convert this into text for display by the PED or vice versa.

In one embodiment, the method operates to convert a spoken announcement from the operator or crew of the passenger vehicle into a text message, which is then displayed on the PED and/or on a remote display assembly to which the PED is connected either wirelessly or by a hard-wired connection. The conversion of the spoken announcement into a text message may include translation into a different language, for example allowing the text message to be displayed in the language selected by the passenger. Conversion of the spoken announcement into a text message and/or translation of the text message into a different language may be performed by the communication system of the vehicle, which broadcasts a text message to the PED of the passenger, or may be performed by the PED, once information containing the spoken announcement is received by the PED from the broadcast system.

In one embodiment, a spoken announcement by the operator or crew of the vehicle is broadcast to the PED of the passenger and is translated into a different language by the PED, prior to being displayed to the passenger by an audio function of the PED. Suitable software for speech translation is known in the art. The PED may also convert the spoken message into a text message and display of the audio message may be accompanied by the display of the text message.

Information transmitted by the broadcast system of the vehicle to the PED of the passenger may be of a variety of different forms and content. In one embodiment, the information received by the PED from the broadcast system may be for entertaining the passenger during the journey. The information received may, for example, enable the PED to provide an audio and/or visual display to the passenger. The communication system may comprise a data storage assembly on which audio and/or visual content are stored, such as films, music and videos. The PED is provided with a suitable interface to allow the passenger to select the entertainment content to be received from the broadcast system and displayed.

More significantly, the present invention allows the operators of the vehicle to communicate directly with the passenger using the PED to transmit information important to the safety, security and comfort of the passenger before, during and after the journey. As discussed hereinbefore, there are a range of messages that are needed to be relayed to passengers during their journey in the vehicle. In particular, in many cases regulations require passengers to be briefed on safety aspects of the vehicle and its operation. Operators or crew members of the vehicle will also need to communicate with passengers in the event of an emergency. In a particularly preferred embodiment, the present invention is operable to have the broadcast system of the vehicle transmit information to the PED of one or more passengers, which triggers the PED to interrupt any audio/visual display underway and to display a message to the passenger. The displayed message may be communicated to the PED by the broadcast system. Alternatively, the information transmitted to the PED triggers the PED to retrieve and display information content, such as a message stored in the memory of the PED In particular, the PED may be arranged to deliver such crew-based communications as required by law to be broadcast in a manner to over-ride any media such as, but not limited to video and audio being digested on the PED.

As discussed hereinbefore, a significant aspect of the present invention is for the broadcast system of the passenger vehicle to communicate with the PED of a passenger and transmit information to the PED to be processed and have the PED take the appropriate action. In one embodiment, the PED of the passenger is operable to communicate with the broadcast system and allow the PED to transmit information to the communication system of the vehicle. In one embodiment, the passenger uses the PED to communicate with the operator of the vehicle, such as a crew member, for example to summon a person for assistance. Alternatively or in addition, the PED may transmit information to the broadcast system of the vehicle regarding its status or the status of the passenger, for example the location of the passenger within or relative to the vehicle.

The PED may store some or all information received from the broadcast system of the vehicle. For example, the PED may store information relating to certain messages to be displayed to the passenger, for example safety messages, such as ‘fasten seat belts’. The PED may then repeatedly display the message to the passenger, until the passenger acknowledges the message and/or takes the appropriate action. An acknowledgement may then be transmitted by the PED back to the broadcast system of the vehicle.

In one embodiment, information received by the PED is automatically deleted at the end of the journey. This may be performed, for example, by a time-based delete function and/or a location-based delete function.

In an alternative embodiment, information received by the PED is stored within the PED for a period of time. Stored information may be encrypted. Stored information may be accessible to others, in particular the operator company of the vehicle, for example by way of a supervisor App designed to retrieve information from the PEDs of passengers.

The communication between the PED of the passenger and the broadcast system of the vehicle occurs when the passenger is within the vehicle. In one embodiment, communication between the PED and the broadcast system of the vehicle may occur when the passenger is outside the vehicle, for example within the vicinity of the vehicle. In such embodiments, communication between the PED and the broadcast system is generally wireless. This allows information to be transmitted to the passenger before they embark, for example to confirm the passenger is entering the correct vehicle or to provide information regarding the embarkation procedure.

In one preferred embodiment, the PED carries out a check to confirm that it is communicating with the correct vehicle for the journey being made by the passenger. Preferably, the PED is operable to ignore signals received from vehicles other than the vehicle intended for travel.

In general, the PED will comprise devices for displaying information, such as a screen for visual displays, for example still images, moving images and/or text, and a loud speaker and/or attachment to personal listening devices, such as headphones and ear pieces, for audio displays. Within the vehicle, the PED may be connected to a remote or external display device of the communication system. The external display device may be any suitable device. In many passenger vehicles, suitable display devices are present at each seat, for example the communication and entertainment system comprised in the seat assembly. The external display may be a video display screen equipped with signal processing, computer graphics, graphical user interface, navigational support i.e. within the commercial aircraft sector a classical IFE screen arrangement.

However, the present invention allows the standard communication and entertainment system installed in the seat assembly to be dispensed with and replaced by a simple monitor or screen. The invention may include communication to simple external display(s), such as monitors or screens, that do not possess the infrastructure commonplace within passenger carrying vehicles, such as signal processing, computer graphics, graphical user interface, support for navigational controller (channel selection, crew call etc.) and audio channel. In one embodiment, this infrastructure is provided by the PED of the passenger. For example, the seat assembly may comprise a simple monitor or screen and audio output for use by the PED as a remote audio/visual display.

In the present specification, references to a ‘simple screen’ or a ‘simple monitor’ are to a screen or monitor that does not possess the infrastructure commonplace within passenger carrying vehicles, such as signal processing, computer graphics, graphical user interface, support for navigational controller (channel selection, crew call etc.) and audio channel, but rather comprises a power supply and only those components necessary to display information content to the passenger under the control of the PED of the passenger.

In one embodiment, the simple monitor comprises a processor, data memory and graphics components to provide the passenger with a simple browser-only function, in order to be able to display information content, such as messages and announcements from the operator or crew of the passenger vehicle, without the need for a connection to a PED. In this way, in the event the passenger is not operating a PED at any time during the journey, essential information, such as safety announcements, can still be provided to the passenger as required.

The simple monitor may comprise a processor and data memory sufficient for it to connect via a browser to a wireless entertainment system or connectivity system, by which the simple monitor may display content from streamed information.

The invention may be presented with one or more methods for connecting a peripheral device such as a PED to the screen, whereby the screen becomes an external or remote display for the PED for displaying the information content delivered to the PED screen. Connection of the PED to the remote audio/visual display may be by any suitable means, for example a wired connection, for example using a cable or a receiver for the PED, or a wireless connection.

The external display in one embodiment may be configured in such a way as to be free from user controls whereby it is in a permanent state of standby until addressed by the PED.

The communication system may comprise an interface. The interface may provide a connection between the PED and the broadcast system. For example, the interface may communicate with the broadcast system by a hard wired connection or a wireless connection, with the PED being connected to the interface in use. The PED may connect to the interface by a hard wired connection, for example by way of a socket provided on or connected to the interface. Alternatively, the PED may connect to the interface by a wireless connection.

In embodiments in which the communication system comprises a display assembly for use by the PED as an external display, the interface may provide a connection between the PED and the external display. Again, this connection may be a hard wired connection or a wireless connection.

The interface may act as a source of electrical power for the PED, for example allowing the PED to be recharged during the journey.

The interface may be located in any suitable position within the vehicle. In one preferred embodiment, the interface is located in a seat assembly to be occupied by a passenger, for example in the back of the seat in front of the passenger or in the frame of the seat assembly.

As described above, in a preferred embodiment of the present invention, a display assembly external to the PED is employed to display information content to the passenger during the journey. In particular, it is possible for the display assembly to consist essentially of a simple screen, the functions for operating the screen being provided by the PED.

In a further aspect, the present invention provides a seat assembly for a passenger vehicle, the seat assembly comprising an audio/visual display assembly, such as a monitor, for displaying audio and visual content to a passenger occupying the seat assembly; and means for connecting a portable electronic device (PED) of the passenger to the audio/visual display assembly, whereby audio and visual content may be displayed by the PED remotely on the audio/visual display assembly.

There is also provided a passenger vehicle comprising a seat assembly as hereinbefore described.

In a still further aspect, the present invention provides a method for displaying audio/visual content to a passenger occupying a seat assembly in a passenger vehicle, the seat assembly comprising an audio/visual display assembly, the method comprising connecting a portable electronic device (PED) of the passenger to the audio/visual display assembly and operating the PED to display audio/visual content on the audio/visual display assembly.

In many embodiments, the audio/visual display assembly comprises a screen or monitor, in particular a monitor mounted in the back of the seat in front of the passenger occupying the seat assembly and facing the passenger. Typically, the audio/visual display assembly also comprises an outlet for audio displays, for example a socket for connecting personal listening devices, such as headphones or earpieces. The audio/visual display assembly comprising the screen or monitor may be incorporated into the structure of the seat, as is known in the art. Alternatively, the audio/visual display assembly may be a separate assembly and added to an existing seat, without requiring modification of the seat assembly.

Within the public transportation environment, the infrastructure to deliver passenger entertainment services is expensive, heavy (in terms of weight) and focused on providing media delivered from within a local server based environment, that is a server located somewhere within the envelope of the passenger carrying vehicle. The passenger seat itself therefore needs to provide connectivity and interface hardware, a screen, a power supply and processing for the display/audio. The typical installation is power hungry, requiring the vehicle to provide a significant amount of power to the seat installations, tends to be heavily engineered and is generally permanently mounted within the seat envelope, as the seat installation needs to satisfy many requirements, such as certification standards, which in turn leads to relatively heavy pieces of equipment. As a result these incumbent systems are both expensive to install and expensive to operate incurring significant maintenance costs and media licensing, such as Digital Rights Management (DRM) to permit the display of most of the content available to the passengers.

The increased weight of the vehicle as a result of the total installation leads to an ongoing negative operational cost due to an increased fuel burn. On-going maintenance, frequent media updates and obsolescence incur further operation costs and issues for the management of the installed equipment. As an example it generally takes more than 2 years to develop a new system for installation within an aircraft; the speed of development in consumer technology inevitably results in delivered on-board systems being behind the standards expected by the travelling public. The average economic installed life for the equipment results in the installation becoming obsolete before its predicted service life expires. In turn the passenger carriers, such as airlines, are required to stock and support equipment that may be increasingly expensive and difficult to source due to the annual product evolution in the consumer marketplace which, due to its volume, determines product and component availability.

In contrast the travelling public have, by and large, adopted a more progressive approach with their personal equipment (PEDs). It is not unreasonable to expect PED's to be replaced after 24 months ownership, in fact within some demographics the swap-out time is 12 months. This ‘tech’ as it is commonly referred to, advances in capability on an annual basis. The screen resolution and clarity, the processing capability, the memory, communication standards and the peripherals supported supersede the ‘fixed’ transport based systems each year that passes. On top of that PED's often supplement traditional office based systems enabling the travelling public to perform many of the duties required of them during a standard business day.

At the same time airlines consistently strive to improve the economics of running an airline. This includes increasing the number of passengers per flight resulting in a reduced seat pitch, reducing fuel burn during flight through weight savings and improvements to the passenger experience such as the provision of power and internet connectivity. Adding connectivity and increasing passenger numbers contradict the economics improvements and results in less space to use traditional portable computers and even the current generation of tablets thereby impacting the passenger experience.

In broad terms, the present invention helps passenger carriers improve economics of the transport system by reducing the infrastructure surrounding the provision of passenger entertainment systems via the adoption of simple video display devices in compliance with regulatory body safety requirements while providing better passenger experiences.

Various embodiments of the present invention will now be described by way of example only having reference to the accompanying drawings, in which:

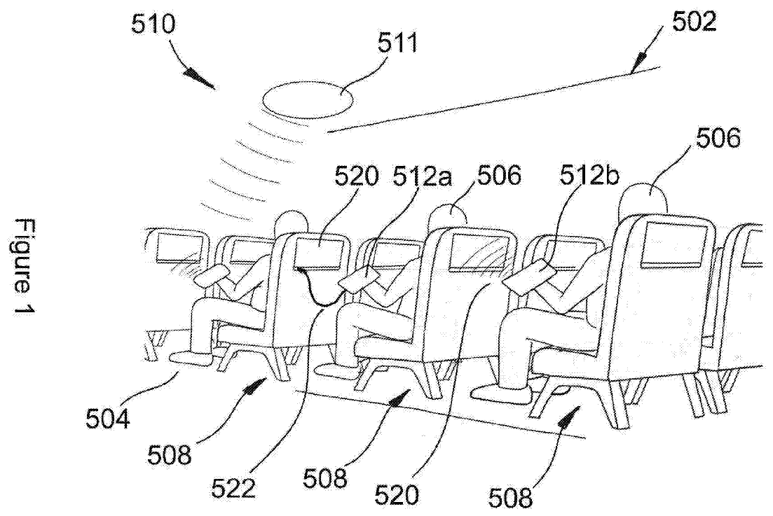

FIG. 1 is a diagrammatical representation of the interior of a passenger vehicle according to one embodiment of the present invention.

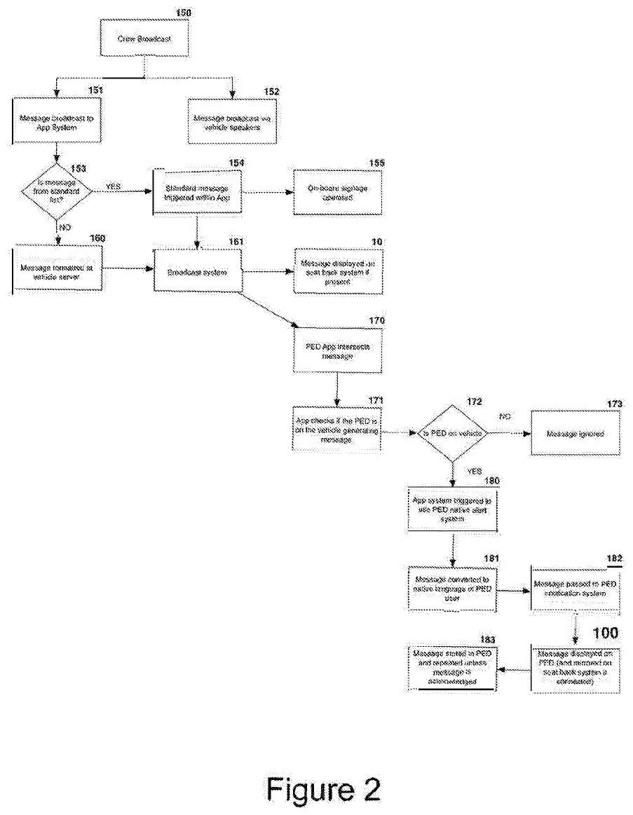

FIG. 2 is a schematic block diagram of a messaging system configured according to some embodiments of the present invention.

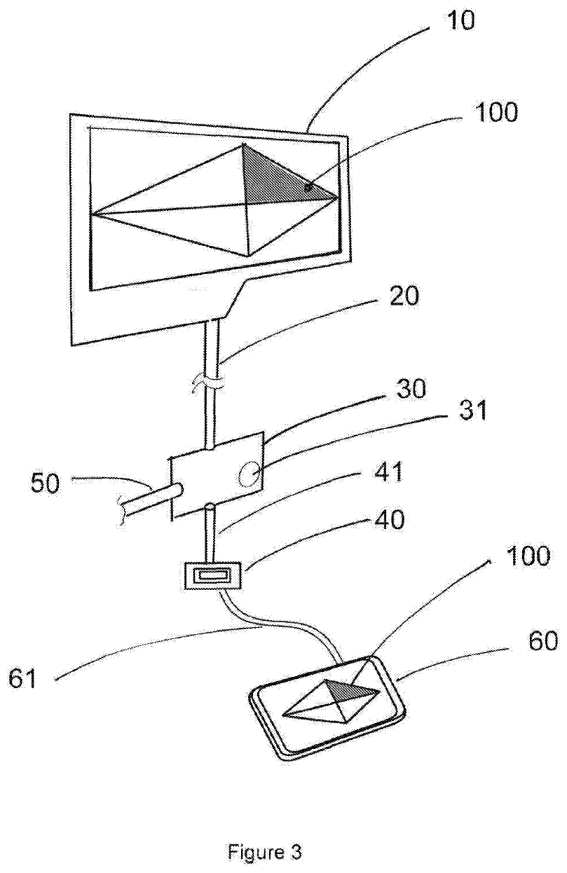

FIG. 3 is a schematic representation of installed system components in one embodiment of the present invention.

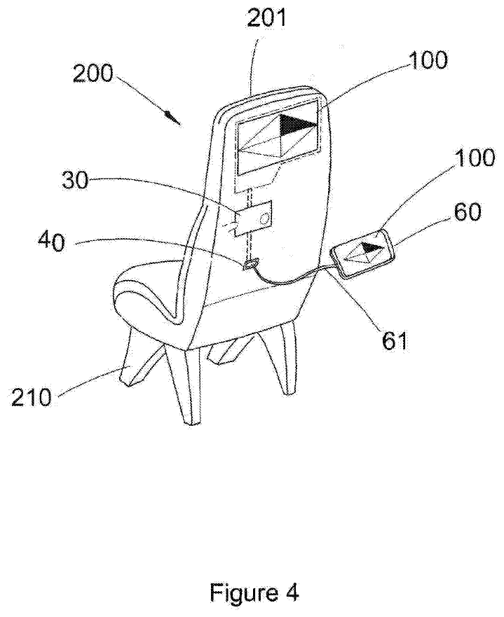

FIG. 4 is a perspective view of the system of the present invention installed in a typical passenger seat.

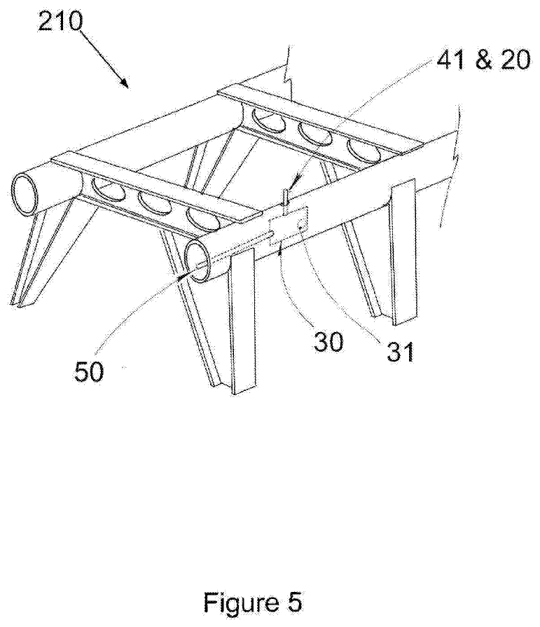

FIG. 5 is a perspective view of a typical seat structure implementation of the present invention.

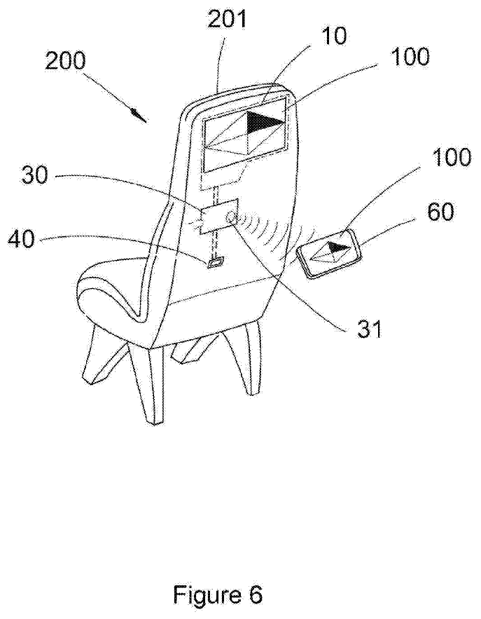

FIG. 6 shows a perspective view of a further embodiment of the installed system of the present invention.

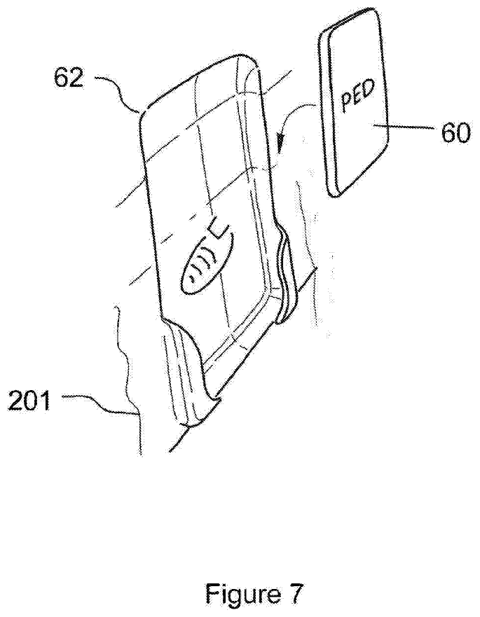

FIG. 7 shows a perspective view of a further embodiment of the present invention showing a pocket for receiving a PED.

Referring to FIG. 1, there is shown a passenger vehicle, generally indicated as 502. The vehicle may be any passenger vehicle in which one or more passengers are conveyed. The present invention is particularly suitable for use with aircraft. However, the passenger vehicle may alternatively be a bus, coach, train, boat or ship.

The vehicle 502 has a compartment 504 in which passengers 506 are generally seated for travel in seat assemblies 508. The vehicle 502 is provided with a communication system, generally indicated as 510, comprising a broadcast system 511 for transmitting information to the passengers 506. The broadcast system 511 communicates wirelessly with a PED 512a, 512b of a passenger. Alternatively, the broadcast system 508 may communicate with the PED 510 by a hard wired connection.

In the embodiment shown in FIG. 1, each seat assembly 508 is provided with an audio/visual display assembly 520, located in the back of the seat in front of each passenger 506. In one arrangement, a PED 512a is connected to the respective display assembly 520 by a cable 522. The cable 522 allows communication between the PED 512a and the display 520, in particular allowing the PED to control the display assembly and display information content, such as entertainment content selected by the passenger from the PED or from a central data storage unit of the communication system 510 and information provided by way of announcements from the crew of the vehicle. The cable 522 may also provide electrical power to the PED, for example to charge the battery of the PED. In an alternative arrangement the PED 512b communicates wirelessly with the display assembly.

Components of embodiments of the communication system 510, the seat assemblies 508 and their operation, together with the operation of the PED are illustrated in FIG. 2 and the subsequent figures and described in detail hereinbelow.

FIG. 2 illustrates a block diagram of the operation of a vehicle messaging system according to some embodiments of the present invention. Prior to commencement of the journey, during the journey and at the end of the journey the crew supporting the vehicles journey are required to make announcements 150.

Announcements may be regular service bulletins, safety bulletins, emergency bulletins or others whereby the crew may wish to communicate with the vehicle passengers. The messages may be codified to reflect operator preferences, to simplify announcement routing or for the crew to select a pre-recorded announcement via a pre-determined control panel button. The crew announcement (message) in one embodiment is routed via an App control system 151 and via audio speakers 152 present within the vehicle as per current convention. The message may be of a standard format pre-defined and stored within the App control system. Logic 153 within the App control system determines if the message is of pre-defined format. If the message is of pre-defined format such as the aircraft-based ‘Fasten Seatbelts’ message, the standard message 154 is retrieved from the App control system 151 and forwarded to the App Broadcast System 161 and signals the vehicle signage system 155 to display the message on the vehicle display units/signs. If the message passed to the Logic system 153 is not of a standard format the message is interpreted by the vehicle-based server 160 and formatted according to the message type. As an example, the message may be for an individual passenger or group of passengers; the message may be a simple acknowledgement for example if a member of the crew has been summoned, the message may be text-based, audio-based or video-based.

In a further embodiment the message may be generated by a passenger to be routed to a crew member or another passenger. The message addressed by the vehicle server 160 would then be routed accordingly by the App Broadcast System 161. In the embodiment shown in FIG. 2 the message is routed to a seat back monitor 10 if present and if it is appropriate to display the message on the seat back monitor and concurrently processed by the App stored and operated within the passengers PED (Portable Electronic Device) 170. The message may be communicated by wireless means or via direct connection of the PED to the vehicle wiring/communication system.

The PED operated App 171 checks the message origin to ensure the message is relevant to the vehicle in which the PED is located. The PED Logic system 172 determines the location of the PED; if the PED is not within the vehicle the message is ignored by the PED based App 173. In this way the system is prevented from alerting passengers on other vehicles. On determination the PED is in the vehicle, the PED App triggers the PED native messaging system 180.

In another embodiment the App may, at the PED users discretion, convert the message to the native language of the PED user 181. Conversion in one aspect may be simply via a code-based system whereby, as an example, Codel relates to Fasten Seatbelts, which then causes ‘attacher la ceinture de securite’ to be displayed on the PED screen of a French speaking person, for example. In another embodiment the code could trigger a voice message in the users native language. In yet another embodiment text to speech software known in the art many convert the message to an audio-based message. Conversely if the message was delivered as an audio message, conversion utilities known in the art may translate the message into the native language as a text display or audio message or both.

The resulting message, converted or not, is passed to the PED notification system 182 at which point the PED in-built notification system delivers the message to the PED screen and seat back monitor 10, if connected. The message is stored within the App and re-displayed using the PED notification system until the PED user acknowledges the message 183.

In another embodiment of the invention, messages are automatically deleted at the end of the journey signified by a time based limit or a location based service. In a further embodiment, the messages may be stored within the App system for a given calendar period. The stored messages being encrypted and only available to offline supervisor Apps designed to retrieve the messages.

With reference to FIG. 3, there are shown the basic components of a seat assembly of one embodiment of the present invention, in which a simple display screen 10 is provided with an incoming communication line 20 delivering power and content 100 hosted by a PED 60 of a passenger. The simple screen 10 provides the necessary equipment to energise the display only. The simple screen receives data from an interface device 30, which in turn is connected to a power supply 50 and to an external input device 40.

In another embodiment, the interface device 30 may be housed within the envelope of the simple display screen 10. The interface device 30 may provide wired links to external devices and may include, but is not limited to Bluetooth, Near Field Communications (NFC), and Wireless Internet (WiFi) communications devices.

On connecting the PED 60 via the socket, 40, or via wireless means 31, the content 100 displayed on the PED 60 is mirrored on the simple display 10.

The simple display 10 may comprise a graphics display technology including, but not limited to TFT, OLED, LCD, DLP, POLED. Alternatively, the simple display may comprise a projected display utilising a Digital Light Projector (DLP) or similar.

In the embodiment shown in FIG. 3, the interface device 30 and the simple screen 10 receive power from the incoming power feed 50. In another embodiment the incoming power feed may also provide a bi-directional data channel whereby data transmitted over the power lines is interpreted by the interface device 30 and broadcast to the PED 60 and hence to the simple display. The incoming data may comprise audio-, text- or video-based images issued by the carrier. Such data may include simple messages such as ‘Fasten Seat Belts’ or other such information the carrier sees fit.

In a further embodiment, the transmitted data may comprise a series of codes to cause predefined messages contained within a data store associated with the interface device to be displayed on the PED 60.

In a still further embodiment, the codes may be transmitted wirelessly to activate content and messages stored within an App provided by the carrier to be displayed on the PED 60 and hence the screen.

In another embodiment the interface device 30 may be configured to deliver power to the connected PED, in order to prolong the consumption of media. The delivered power may deliver charge to the PED 60 without screen sharing. The delivered power may also be configured to charge other portable devices such as, but not limited to, cameras and games devices/consoles.

Turning to FIG. 4, there is shown an arrangement incorporated into, or attached to a seat assembly 200 of a passenger carrying vehicle. The seat assembly 200 comprises a seat frame 210 and a seat 201. The seat assembly 200 in one embodiment has the simple screen 10 embedded within the body of the seat 201. The interface device 30 is located within a void in the seat structure, so as to simplify the build of the seat and also to maximise the structural integrity of the seat and the seat structure itself. The connection to the PED 60 may be via a permanently connected cable 61, for example a pigtail emanating from the seat back. The connection may include a seat back mounted socket 40 of the type known in the art.

The connection 61 may comprise any suitable cable, such as a commercial off-the-shelf cable. The connection may comprise a USB to USB cable. The USB specification for the communication is preferably USB 3.0 or higher. At the physical device level, the USB socket could be a USB-C connector or later generation.

The connection socket 40 may be a display port or later generation high density/definition standard compatible interface.

The connection between the PED 60 and the display 30 may be facilitated by a separate interface device 30. The simple display 10 in another embodiment may be remotely situated from the interface device 30.

The interface device 30 may be housed within the simple screen 10 envelope or integrated directly onto the simple display electronic hardware.

The interface device 30 may interface directly to the appropriate vehicle power system.

The interface device 30 may communicate with other devices within the passenger carrying vehicle through the power system to which it is connected. The communications over the power system may include seat number, location or other unique identity that may facilitate location-based services. Location based services may include but are not limited to attendant call/cancel, seat-to-seat instant messaging, crew to seat messaging, and seat to crew messaging. Messaging may be voice-, text- or video-based or any combination of these.

The interface device 30 of another embodiment may include such hardware as to provide passenger monitoring for feedback to a crew-based dashboard. Passenger monitoring may include but not limited to passenger temperature, zone temperature, passenger wake state and general passenger wellness.

The interface device 30 may deliver power to the simple display 10.

The interface device 30 may be required to perform power conversion from incoming power to that required of connected devices, such as the PED 60.

The interface device 30 may deliver power to one or more discrete devices such as a PED. The interface device 30 may be connected to sockets 40 mounted ergonomically to provide charge to a connected PED, mobile computer, camera, portable game console and the like.

In a further embodiment the sockets 40 for portable devices may also be enabled to transmit data.

In a further embodiment the interface device 30 may be located within the seat frame 210.

The interface device in another embodiment may be configured in such a way as to utilise the supporting structure of the seat or bulkhead for packaging, protection and heat dissipation.

Referring to FIG. 5, there is shown an alternative arrangement for the interface device 30 within the seat assembly. As shown, the interface device 30 is located within a seat frame 210. The power supply 50 enters the frame 210 and connects to the interface device 30. Connections 20 and 41 are routed within the seat structure to connect the simple display screen 10 and any peripheral socket 40 to the interface device 30. In such an arrangement the seat frame 210 may provide improved thermal management via direct contact with metal elements of the seat frame. Furthermore, locating the interface device within the seat frame 210 facilitates a reduction in complexity and weight by reducing the cable loom length and unnecessary packaging of the interface unit 30, as the seat frame provides a resilient enclosure.

With reference to FIG. 6, there is shown a further embodiment of a seat assembly 200 incorporating the present invention. The general arrangement is the same as shown in FIG. 4 and discussed above. However, in the embodiment shown in FIG. 6, the connection of the PED 60 to the simple display screen 10 is facilitated by wireless connectivity 31 to the interface device 30.

In another embodiment, the simple display 10 may be mounted directly within the furniture 200 of a passenger carrying installation whereby the simple display is considered as an integral part of the installed furniture.

In alternative embodiment, the simple display 10 may be arranged as a removable device, whereby it can be replaced in routine maintenance without the recourse to special tools, that is using tools regularly found in a consumer tool box.

The simple display 10 may be considered to be a structural component of the passenger seat 201. Alternatively, the simple display 10 may form the seat-back.

The simple display 10 may be deployed within an assembly to enable the display to be articulated to provide a more convenient viewing angle for the person consuming the media presented on the simple display. Such assemblies are known in the art.

In a further embodiment, the simple display 10 is mounted on a structure capable of being stowed away in compliance with relevant regulations for the operation of the vehicle, such as, in the case of aircraft travel, take-off and landing.

Finally, referring to FIG. 7, there is shown details of one embodiment of the present invention. In the embodiment shown in FIG. 7, the installation comprises a pocket 62 that receives the PED 60. The pocket 62 may be in a region of the seat 201 not occupied by a display screen, such as the seat back, seat arm, fold down table or within the voids immediately adjacent the seat, such as between the seat arm mechanism and the seat back, between the seat bottom and the floor supporting the seat 201.

In a further embodiment the pocket 62 may be part of the display screen 10 mounting apparatus.

The pocket 62 may be configured to provide charging for the PED 60, as known in the art, with the PED communicating with the broadcast system and/or the display screen 10 wirelessly.

In an alternative embodiment, the pocket 62 may provide a hard wired connection between the PED 60 and the broadcast system and/or the display screen 10, when the PED 60 is located in the pocket.

In embodiments of the present invention, the power sockets 40 may provide wireless communications such as, but not limited to, Bluetooth and IEEE 802 based wireless standards. For example, the wireless communications may include Bluetooth Mesh, Bluetooth Low Energy, or RFID. In such embodiments, the PED 60 has a hard wired connection with the power sockets 40.

Alternatively, the interface device 30 may contain the wireless communications apparatus 31, including the necessary antenna arrangements. Again, in such embodiments, the PED 60 may have a hard wired connection with the interface device 30.

1-41. (canceled)

42. A method for communicating with a passenger making a journey in a vehicle, the vehicle having a broadcast system for transmitting information to one or more passengers, the passenger having and operating a Portable Electronic Device (PED), the PED operable to receive information from the broadcast system of the vehicle and to display information content to the passenger;
the method comprising transmitting information to the PED of one or more passengers within the vehicle;
wherein the PED displays information content derived from the information transmitted to the PED.

43. The method according to claim 42, wherein the PED displays information content comprised in the information transmitted by the broadcast system.

44. The method according to claim 42, wherein the PED displays information content retrieved from the memory of the PED; the display of the information content being triggered by information broadcast to the PED by the broadcast system.

45. The method according to claim 42, comprising converting information using speech-to-text operations, wherein the PED displays a text message to the passenger.

46. The method according to claim 42, comprising translating information from a first language to be displayed by the PED in a second language.

47. The method according to claim 42, wherein the PED of the passenger is displaying audio and/or visual content; transmission of information from the broadcast system to the PED causing the PED to interrupt the audio and/or visual display and display information content derived from the transmitted information.

48. The method according to claim 42, wherein information is deleted from the PED after the passenger has completed a journey in the passenger vehicle or wherein information is stored in the PED after the passenger has completed a journey in the passenger vehicle.

49. The method according to claim 42, wherein information is transmitted to the PED during one or both of while the passenger is within the passenger vehicle or while the passenger is outside the passenger vehicle.

50. The method according to claim 42, wherein the PED conducts a check to confirm the communication is with the correct passenger vehicle.

51. The method according to claim 42, wherein the PED displays information content on the PED and/or displays content on a display assembly remote from the PED wherein the remote display assembly is comprised in the communication system of the passenger vehicle, wherein the remote display assembly is comprised in a seat assembly of the passenger vehicle.

52. A communication system for a passenger vehicle, the communication system comprising a broadcast system for transmitting information to one or more passengers travelling in the vehicle, the one or more passengers having and operating a Portable Electronic Device (PED), the PED operable to receive transmitted information from the broadcast system of the vehicle and to display information content to the one or more passengers.

53. The communication system according to claim 52, further comprising a display assembly, in use the PED displaying information content on the display assembly.

54. The communication system according to claim 52, further comprising:
an interface;
wherein the interface provides a connection between the broadcast system and the PED of a passenger;
wherein the interface is connected wirelessly to the broadcast system and/or the interface is hard-wired to the broadcast system; and/or
wherein the interface is wirelessly connectable and/or connectable by a hard wire connection to the PED of a passenger.

55. A seat assembly for a passenger vehicle, the seat assembly comprising:
an audio/visual display assembly for displaying audio and visual content to a passenger occupying the seat assembly; and
means for connecting a portable electronic device (PED) of the passenger to the audio/visual display assembly, whereby audio and visual content may be displayed by the PED remotely on the audio/visual display assembly.

56. The seat assembly according to claim 55, wherein the audio/visual display assembly is comprised in a seat.

57. The seat assembly according to claim 55, wherein the audio/visual display assembly is applied to a seat of the seat assembly.

58. The seat assembly according to claim 55, further comprising an interface for connecting the audio/visual display assembly to the PED.

59. The seat assembly according to claim 58, wherein the connection between the audio/visual display assembly and the PED is wireless and/or hard-wired.

60. The seat assembly according to claim 55, wherein the audio/visual display assembly comprises a simple screen or simple monitor.
